(19) United States
(12) Patent Application Publication
(10) Pub. No.: US 2024/0398389 A1
(43) Pub. Date: Dec. 5, 2024

(54) ULTRASONIC DIAGNOSTIC DEVICE AND STORAGE MEDIUM

(71) Applicant: GE Precision Healthcare LLC, Waukesha, WI (US)

(72) Inventor: Shunichiro Tanigawa, Hino (JP)

(21) Appl. No.: 18/675,782

(22) Filed: May 28, 2024

(30) Foreign Application Priority Data
May 29, 2023 (JP) 2023-088273

(51) Int. Cl. A61B 8/00; A61B 8/08; A61B 8/14

(52) U.S. Cl. CPC A61B 8/585; A61B 8/14; A61B 8/461; A61B 8/5207

(57) ABSTRACT

A method of controlling an ultrasonic probe includes: setting conditions of an ultrasonic probe for acquiring ultrasonic images of the subject; transmitting an ultrasonic beam from the ultrasonic probe towards the subject; receiving, by the ultrasonic probe, an echo of the ultrasonic beam from the subject in accordance with the conditions; generating an ultrasonic image of the subject based on the echo received by the ultrasonic probe; and creating input images to be input to the trained model based on the ultrasonic images displayed on the display unit. Each input image created by the processor is created such that the length of the subject in the depth direction is a first length, and the length of the subject in a direction perpendicular to the depth direction is a second length.

### CROSS-REFERENCE TO RELATED APPLICATION

This application claim priority to Japanese Patent Application No. 2023-088273, which was file on May 29, 2023 at the Japanese Patent Office. The entire contents of the above-listed application are incorporated by reference herein in their entirety.

### TECHNICAL FIELD

The present disclosure relates to a diagnostic ultrasonic device capable of changing imaging conditions, and a storage medium containing commands to be executed by the diagnostic ultrasonic device.

### BACKGROUND

When scanning a subject using an ultrasonic diagnostic device, the user sets the imaging conditions for each imaging site before starting to scan the subject.

Imaging conditions include a variety of parameters. Therefore, a user may have difficulty selecting optimal parameters for each imaging site. Therefore, ultrasonic diagnostic devices are prepared with preset conditions that define the imaging conditions for each imaging site in advance. When imaging a subject, the user can select preset conditions corresponding to the imaging conditions of the subject in order to set the imaging conditions corresponding to the imaging site.

However, it is often difficult for some users to perform an examination of a subject under appropriate imaging conditions because they may not be able to select appropriate preset conditions or may not be able to fully execute parameter adjustments according to the imaging site.

As a method for resolving this problem, a technique is being considered that uses deep learning technology to determine the imaging site of the subject based on the ultrasonic image of the subject, and automatically changes the imaging conditions if the current imaging conditions set by the user are not appropriate for the imaging site of the subject.

When deducing the imaging site of a subject, an input image is created based on the ultrasonic image of the subject, and the input image is input to a trained neural network to deduce the imaging site.

However, depending on the ultrasonic viewing angle and the ultrasonic depth, the deduced imaging site may not match the actual imaging site. In this case, if the imaging conditions are automatically changed, there is a risk that the imaging conditions will be changed to those that are not appropriate for the actual imaging site.

Therefore, technology that can improve the accuracy of identifying the imaging site is desired.

### SUMMARY

According to an aspect, an ultrasonic diagnostic device, may include including: an ultrasonic probe; a display; and a processor communicating with the ultrasonic probe and the display; wherein the processor performs: setting conditions for acquiring ultrasonic images of the subject; transmitting an ultrasonic beam to the ultrasonic probe and causing the ultrasonic probe to receive an echo from the subject in accordance with the conditions, and generating an ultrasonic image of the subject based on the echo received by the ultrasonic probe; and creating input images to be input to the trained model based on the ultrasonic images displayed on the display, each input image created by the processor is created such that the length of the subject in the depth direction is a first length, and the length of the subject in a direction perpendicular to the depth direction is a second length.

According to an aspect, a recording medium may store commands executable by a processor, wherein the commands cause the processor to perform: setting conditions for acquiring ultrasonic images of the subject; transmitting an ultrasonic beam to the ultrasonic probe and causing the ultrasonic probe to receive an echo from the subject in accordance with the conditions, and generating an ultrasonic image of the subject based on the echo received by the ultrasonic probe; and creating input images to be input to the trained model based on the ultrasonic images displayed on the display; wherein each input image created by the processor is created such that the length of the subject in the depth direction is a first length, and the length of the subject in a direction perpendicular to the depth direction is a second length.

According to an aspect, each input image may be created so as to have a first length in the depth direction of the subject and a second length in a direction orthogonal to the depth direction of the subject. Thus, regardless of the shape and size of the ultrasonic image used to create the input image, the input image is created to have a predetermined first and second length. Thereby, a common input image size can be achieved, which improves the accuracy when deducing the imaging site.

### BRIEF DESCRIPTION OF DRAWINGS

FIG. 1 is a diagram illustrating a state of scanning a subject via an ultrasonic diagnostic device 1 according to an embodiment.

FIG. 2 is a block diagram of the ultrasonic diagnostic device 1 according to an embodiment.

FIG. 3 is a schematic view of an original image according to an embodiment.

FIG. 4 is an explanatory diagram of the original image P1 according to an embodiment.

FIG. 5 is an explanatory diagram of the original image P2 according to an embodiment.

FIG. 6 is an explanatory diagram of the method of creating the training image PA1 from the original image P1 according to an embodiment.

FIG. 7 is an explanatory diagram of preprocessing according to an embodiment.

FIG. 8 is an explanatory diagram of the method of creating the training image PA2 from the original image P2 according to an embodiment.

FIG. 9 is an explanatory diagram of preprocessing according to an embodiment.

FIG. 10 is a schematic diagram of the original images P1 to Pn and the training images PA1 to PAn created by preprocessing the original images P1 to Pn according to an embodiment.

FIG. 11 is an explanatory diagram of the correct data according to an embodiment.

FIG. 12 is an explanatory diagram of a method for creating a trained model 31 according to an embodiment.

FIG. 13 is a diagram depicting an example of a flowchart executed during an examination of a subject according to an embodiment.

FIG. 14 is an explanatory diagram of step ST11 according to an embodiment.

FIG. 15 is a diagram depicting the next new subject 53 being examined according to an embodiment.

FIG. 16 is an explanatory diagram of step ST11 according to an embodiment.

FIG. 17 is a diagram depicting a variation of the blank region according to an embodiment.

FIG. 18 is a diagram depicting a variation of the blank region according to an embodiment.

FIG. 19 is a diagram depicting a variation of the blank region according to an embodiment.

FIG. 20 is a diagram depicting a variation of an extracted image according to an embodiment.

### DETAILED DESCRIPTION

An embodiment of the present disclosure will be described below; however, the present disclosure is not limited to the following embodiments.

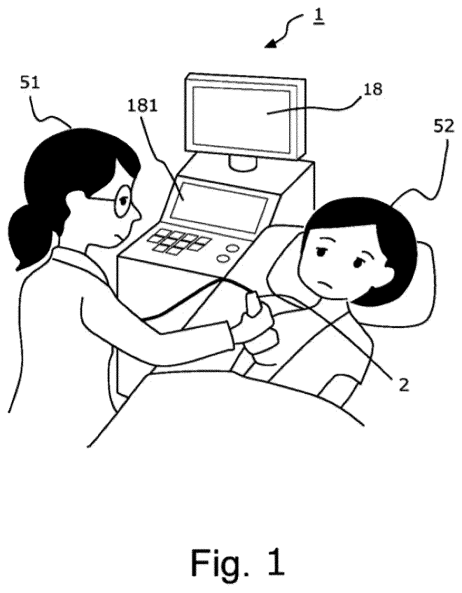

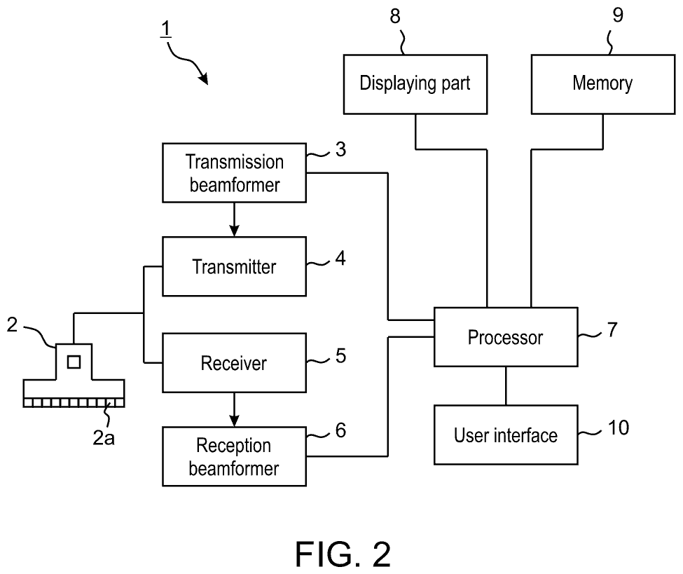

FIG. 1 is a diagram illustrating an aspect of scanning a subject via an ultrasonic diagnostic device 1 according to an embodiment, and FIG. 2 is a block diagram of the ultrasonic diagnostic device 1 according to an embodiment.

The ultrasonic diagnostic device 1 has an ultrasonic probe 2, a transmission beamformer 3, a transmitting apparatus 4, a receiving apparatus 5, a reception beamformer 6, a processor 7, a display unit 8, a memory 9, and a user interface 10. The ultrasonic diagnostic device 1 is one example of the ultrasonic image display system.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating element 2a are reflected in a subject 52 (see FIG. 1) and a reflection echo is received by the vibrating element 2a. The vibrating elements 2a convert the received echo to an electrical signal and output this electrical signal as an echo signal to the receiver 5. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or more processors, including one or more: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

The processor 7 controls the transmission beamformer 3, the transmitter 4, the receiver 5, and the reception beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display unit 8. The processor 7 can process echo data to generate an ultrasonic image. The term “electronic communication” may be defined to include both wired and wireless communications. The processor 7 may include a central processing unit (CPU) according to one embodiment. According to another embodiment, the processor 7 may include one or more processor, another electronic component that may perform a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not illustrated in the drawings) that demodulates RF data. In another embodiment, demodulation may be executed in an earlier step in the processing chain.

Moreover, the processor 7 may generate various ultrasonic images (for example, a B-mode image, color Doppler image, M-mode image, color M-mode image, spectral Doppler image, elastography image, TVI image, strain image, and strain rate image) based on data obtained by processing via the reception beamformer 6. In addition, one or a plurality of modules can generate these ultrasonic images.

An image beam and/or an image frame may be saved, and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasonic images may be read from the image memory and displayed on the display unit 8.

In the present Specification, the term “image” can broadly indicate both a visual image and data representing a visual image. Furthermore, the term “data” can include raw data, which is ultrasonic data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display unit 8 include an LED (Light Emitting Diode) display, an LCD (Liquid Crystal Display), and an organic EL (Electro-Luminescence) display. The display unit 8 displays an ultrasonic image. In the first embodiment, the display unit 8 includes a display monitor 18 and a touch panel 181, as illustrated in FIG. 1. However, the display unit 8 may be configured with a single display rather than the display monitor 18 and the touch panel 181. Moreover, two or more display devices may be provided in place of the display monitor 18 and the touch panel 181.

The memory 9 is any known data storage medium. In one example, the ultrasonic image display system includes a non-transitory storage medium and a transitory storage medium. In addition, the ultrasonic image display system may also include a plurality of memories. The non-transitory storage medium is, for example, a non-volatile storage medium such as a Hard Disk Drive (HDD) drive, a Read-Only Memory (ROM), etc. The non-transitory storage medium may include a portable storage medium such as a CD (Compact Disk) or a DVD (Digital Versatile Disk). A program executed by the processor 7 is stored in the non-transitory storage medium. The transitory storage medium is a volatile storage medium such as a Random-Access Memory (RAM).

The memory 9 stores one or more commands that can be executed by the processor 7. One or more commands cause the processor 7 to execute various types of operations.

Note that the processor 7 may also be configured so as to be able to connect to an external storing device 15 by a wired connection or a wireless connection. In this case, the command(s) causing execution by the processor 7 can be distributed to both the memory 9 and the external storing device 15 for storage.

The user interface 10 can receive input from a user 51 (for example, an operator). For example, the user interface 10 receives instruction or information input by the user 51. The user interface 10 is configured to include a keyboard (keyboard), a hard key (hard key), a trackball (trackball), a rotary control (rotary control), a soft key, and the like. The user interface 10 may include a touch screen that displays a soft key or the like.

The ultrasonic diagnostic device 1 is configured as described above.

When scanning a subject using an ultrasonic diagnostic device, the user sets the imaging conditions for each imaging site before starting to scan the subject.

Imaging conditions include a variety of parameters. Therefore, a user may have difficulty selecting optimal parameters for each imaging site. Therefore, ultrasonic diagnostic devices are prepared with preset conditions that define the imaging conditions for each imaging site in advance. When imaging a subject, the user can select preset conditions corresponding to the imaging conditions of the subject in order to set the imaging conditions corresponding to the imaging site.

However, it is often difficult for some users to perform an examination of a subject under appropriate imaging conditions because they may not be able to select appropriate preset conditions or may not be able to fully execute parameter adjustments according to the imaging site.

As a method for resolving this problem, a technique is being considered that uses deep learning technology to determine the imaging site of the subject based on the ultrasonic image of the subject, and automatically changes the imaging conditions if the current imaging conditions set by the user are not appropriate for the imaging site of the subject.

When deducing the imaging site of a subject, an input image is created based on the ultrasonic image of the subject, and the input image is input to a trained neural network to deduce the imaging site.

However, depending on the ultrasonic viewing angle and the ultrasonic depth, the deduced imaging site may not match the actual imaging site. In this case, if the imaging conditions are automatically changed, there is a risk that the imaging conditions will be changed to those that are not appropriate for the actual imaging site.

Therefore, the ultrasonic diagnostic device 1 of the first embodiment is configured to improve the deducing accuracy of the imaging site to address the above problem. The first embodiment is described below in detail.

Note that in the first embodiment, a trained model is used to deduce the imaging site of the subject, and based on the result of this deduction, a determination is made as to whether the imaging conditions should be changed. Therefore, in the first embodiment, a training phase is performed to generate a trained model, which is suitable for deducing the imaging site of the subject. Therefore, first, a training phase for generating this trained model is described below. Furthermore, after describing the training phase, the method for automatically changing the imaging conditions during the examination of the subject will be described.

### (Training Phase)

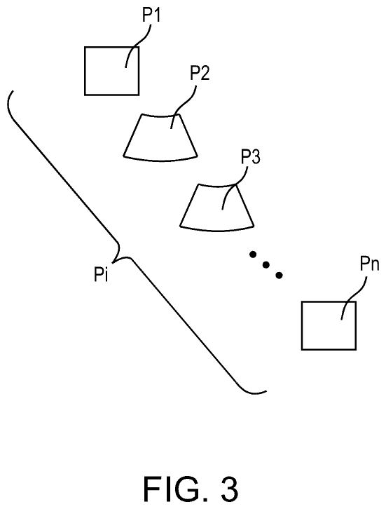

FIGS. 3 to 12 are explanatory diagrams of the training phase.

In the training phase, first, original images are prepared which form a basis for generating the training image.

FIG. 3 is a schematic view of the original images P1 to Pn.

In the present embodiment, ultrasonic images Pi (i=1 to n) are prepared as original images. Ultrasonic images Pi include ultrasonic images acquired at hospitals and other medical facilities, as well as ultrasonic images acquired by medical equipment manufacturers. For example, 5,000 to 10,000 examples of original images are prepared.

The original images P1 to Pn include images of various parts of the body subject to examination. Sites subject to examination include, for example, the “abdomen”, “breast”, and “kidney”, but are not limited to these sites, and a variety of sites subject to ultrasonic examination can be used as the sites to be examined.

Each original image is described below.

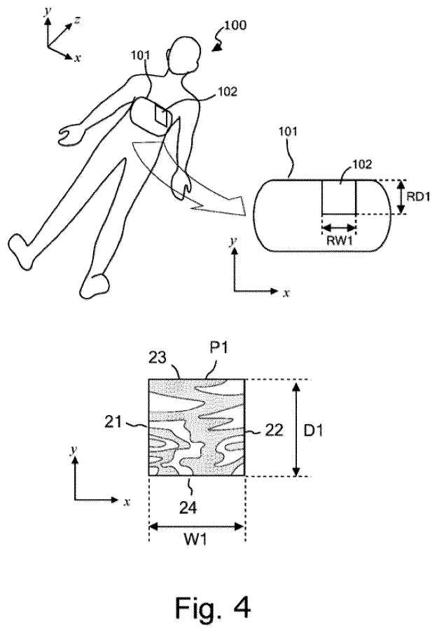

FIG. 4 is an explanatory diagram of the original image P1.

FIG. 4 depicts the subject 100 and the original image P1 obtained by imaging the subject 100.
The upper part of FIG. 4 depicts the subject 100. An enlarged view of a cross-section 101 of the subject 100 is depicted on the right side of the subject 100. Region 102 is depicted in cross-section 101. This region 102 represents a cross-section of a breast of the subject. The region 102 is a square-shaped region. The vertical length RD1 (cm) of the region 102 represents the length of the subject 100 in the depth direction (y-direction). Furthermore, the horizontal length RW1 (cm) of the region 102 represents the length in a direction orthogonal to the depth direction of the subject 100 (width direction of the subject 100).

The lower part of FIG. 4 depicts a schematic diagram of the original image P1 of the region 102.

The original image P1 is square-shaped and has four sides 21, 22, 23, and 24. The vertical length D1 (cm) of the original image P1 represents the length of the subject 100 in the depth direction (y direction) (in other words, length RD1 of region R1). Furthermore, the horizontal length W1 (cm) of the original image P1 represents the length of the subject 100 in the direction orthogonal to the depth direction (width direction of the subject 100) (in other words, length RW1 of region R1). Thus, the length D1 of the original image P1 represents the length RD1 of the region 102, and the length W1 of the original image P1 represents the length RW1 of the region 102. The length D1 of the original image P1 (length RD1 of region 102) is, for example, 4 (cm), and the length W1 of the original image P1 (length RW1 of region 102) is, for example, 4 (cm).

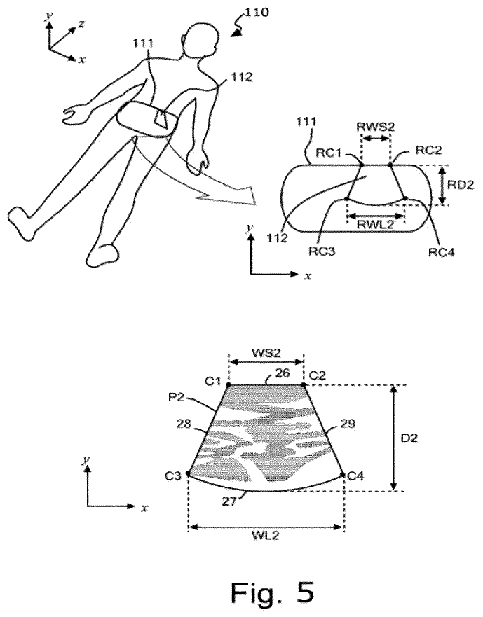

FIG. 5 is an explanatory diagram of the original image P2.

FIG. 5 depicts the subject 110 and the original image P2 obtained by imaging the subject 110.
The subject 110 is depicted in the upper part of FIG. 5. An enlarged view of a cross-section 111 of the subject 110 is depicted on the right side of the subject 110. Region 112 is depicted in the cross-section 111. This region 112 is a cross-section of the kidney of the subject. The region 112 is an essentially trapezoidal-shaped region. The vertical length RD2 (cm) of the region 112 represents the length of the subject 110 in the depth direction (y-direction). Furthermore, the length RWS2 (cm) between the corners RC1 and RC2 of the region 112 represents the length of the upper side of the subject in the direction orthogonal to the depth direction (width direction of the subject). Furthermore, the length RWL2 (cm) between the corners RC3 and RC4 of the region 112 represents the length of the lower side of the subject in the width direction.

The lower part of FIG. 5 depicts a schematic diagram of the original image P2 of the region 112.

The original image P2 has an essentially trapezoidal shape. The original image P2 has four edges 26, 27, 28, and 29. The edges 26, 28, and 29 are straight lines, while the edge 27 has the shape of an arc. The vertical length D2 (cm) of the original image P2 represents the length of the subject in the depth direction (y-direction) (in other words, the length RD2 of the region 112). The length WS2 (cm) between corners C1 and C2 of the original image P2 represents the length RWS2 of the region 112 of the subject 110. Furthermore, the length WL2 (cm) between corners C3 and C4 of the original image P2 represents the length RLS2 of the region 112 of the subject 110. Thus, the length D2 of the original image P2 is the length RD2 of the region 112; the length WS2 of the original image P2 is the length RWS2 of the region 112; and the length WL2 of the original image P2 is the length RWL2 of the region 112. The length D2 of the original image P2 (length RD2 of region 112) is, for example, 10 (cm); the length WS2 of the original image P2 (length RWS2 of region 112) is, for example, 5 (cm); and the length WL2 of the original image P2 (length RWL1 of region 112) is, for example, 10 (cm).

Similarly, ultrasonic images obtained by imaging various sites of various subjects are prepared as original images. A square-shaped original image is depicted in FIG. 4, and an essentially trapezoidal original image is depicted in FIG. 5, but various other shapes of ultrasonic images (for example, fan-shaped) obtained by ultrasonic examination can be used as original images.

These original images P1 to Pn are used to create a training image. The following describes how to create a training image from the original images.

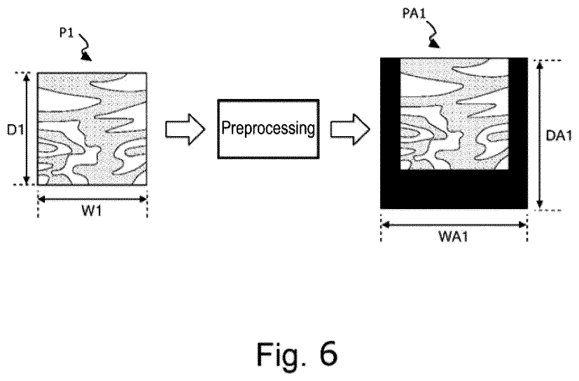

FIG. 6 is an explanatory diagram of the method of creating the training image PA1 from the original image P1.

In the present embodiment, the training image PA1 is created by preprocessing the original images P1.
As described earlier, the original image P1 is square-shaped, and the original image P1 has a size of D1=W1=4 cm. On the other hand, the training image PA1 is square-shaped like the original image P1, but the training image PA1 has a larger size than the original image P1. In the present embodiment, the training image PA1 has a size of DA1=WA1=6 cm, but the size of the training image is not limited to 6 cm, and may be smaller than 6 cm, or larger than 6 cm.

In the present embodiment, preprocessing is performed to create a training image (DA1=WA1=6 cm) from the original image P1 (D1=W1=4 cm). Preprocessing is described below. Note that this preprocessing can be performed by a device that has image processing functions, such as an ordinary computer.

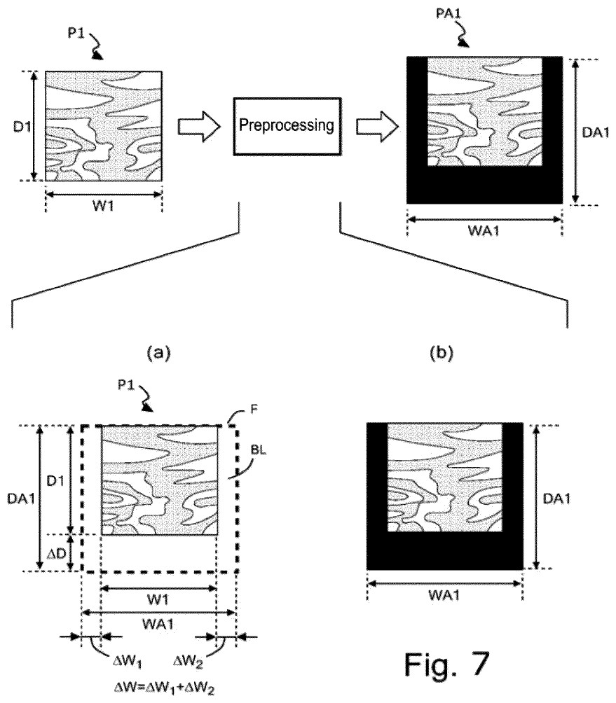

FIG. 7 is an explanatory diagram of preprocessing.

Schematic diagrams (a) and (b) are depicted in FIG. 7 in order to describe the pretreatment process.
First, a schematic diagram (a) is described.
Schematic diagram (a) depicts the contour F of the training image PA1 and the original image P1. The contour F is indicated by a dashed line. The contour F is depicted so that the upper edge of the contour F coincides with the upper edge of the original image P1.
The original image P1 has a length D1 of 4 cm in the depth direction of the subject. The original image P1 also has a length W1 of 4 cm in the width direction of the subject. Therefore, the depth direction length D1 of the original image P1 is AD (=3 cm) shorter than the depth direction length DA1 of the training image, and furthermore, the width direction length W1 of the original image P1 is AW (=AW1+AW2) (AW=3 cm) shorter than the width direction length WA1 of the training image. Therefore, in order to solve the insufficient portion AD1 in the depth direction of the original image P1 and the insufficient portion AW in the width direction of the original image P1, a zero-fill process is performed on the original image P1, filling the blank region BL around the original image P1 with zero data to achieve the training image size. The image after the zero-fill process is performed on the original image P1 is depicted in the schematic diagram (b). In the schematic diagram (b), the zero-filled blank region BL is depicted as a black-filled region. Although the size of the original image P1 is smaller than the size of the training image, the size of the original image P1 can be made to match the size of the training image PA1 by performing the above zero-fill process as a preprocessing step on the original image P1. Note that in the present embodiment, a training image PA1 of the desired size is created by performing the zero-fill process. However, if the training image can have the desired size, a process other than the zero-fill process may be performed.

Other preprocessing is performed as necessary before or after the zero-fill process is performed on the original image P1, but the description of other preprocessing is omitted here.

In this manner, training image PA1 can be created from the original image P1.
Next, an example of creating a training image based on the original image P2 is described.

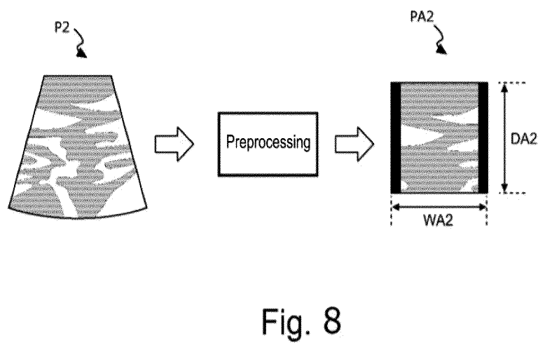

FIG. 8 is an explanatory diagram of the method of creating the training image PA2 from the original image P2.

As described earlier, the original image P2 has an essentially trapezoidal shape. On the other hand, training image PA2 is the same square and the same size as training image PA1 described earlier (DA2=WA2=6 cm).

In the present embodiment, preprocessing is performed to create a training image (DA2=WA2=6 cm) from the original image P2 (essentially trapezoidal shape). Preprocessing is described below.

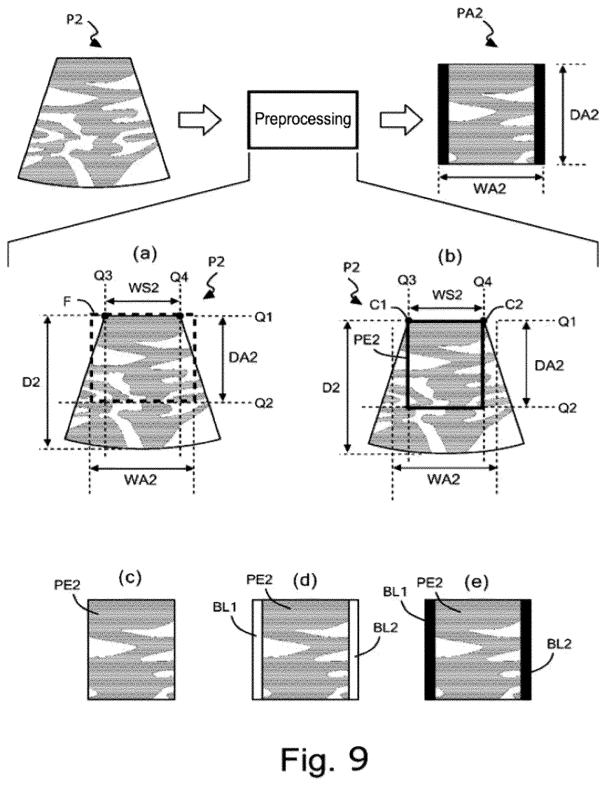

FIG. 9 is an explanatory diagram of preprocessing.

Schematic diagrams (a) to (e) are depicted in FIG. 9 in order to describe the pretreatment process.
First, a schematic diagram (a) is described.
Schematic diagram (a) depicts the contour F of the training image PA2 and the original image P2. The contour F is indicated by a dashed line. The contour F is depicted so that the upper edge of the contour F coincides with the upper edge of the original image P2.

The original image P2 has a length D2 of 10 cm in the depth direction of the subject, and the length WS2 of the upper edge of the original image P2 is 5 cm. Thus, in the case of the original image P2, the length WS2 on the upper edge is 2 cm shorter than the width WA2 of the training image, but the length D2 in the depth direction is 3 cm longer than the length DA2 in the depth direction of the training image. Therefore, a portion of the original image P2 that is suitable for the training image is extracted.

Extracting a suitable portion of the training image from the original image P2 is depicted in schematic diagram (b).

The length D2 in the depth direction of the original image P2 is longer than the length DA2 of the training image. Therefore, with respect to the depth direction of the subject, the region from the position Q1 on the body surface of the original image P2 to the position Q2, which is 6 cm lower in the depth direction, is the image portion used for the training image.

On the other hand, the length WS2 of the upper edge of the original image P2 is shorter than the length WA2 of the training image, so the region from position Q3 in the upper left corner C1 to position Q4 in the upper right corner C2 of the original image P2 is the image portion used for the training image.

Therefore, the region enclosed by positions Q1, Q2, Q3, and Q4 in the original image P2 is extracted as the image portion PE2 to be used for the training image. Schematic diagram (c) depicts the image PE2 extracted from the original image P2 (hereinafter referred to as the “extracted image”).

Next, a zero-fill process is executed on the extracted image PE2 to fill the blank regions BL1 and BL2 along the side edges of the extracted image PE2 with zero data to match the size of the training image. Schematic diagram (d) depicts the extracted image PE2 before the zero-fill process is performed, and schematic diagram (e) depicts the extracted image PE2 after the zero-fill process is performed. Zero-filled regions are depicted as black-filled regions. Therefore, although the size of the extracted image PE2 obtained from the original image P2 is smaller than that of the training image, the training image PA2 can be created from the original image P2 by performing the above zero-fill process as preprocessing. Note that in the present embodiment, the training image PA2 of the desired size is created by performing the zero-fill process. However, if the training image can have the desired size, the training image may be created by performing a preprocessing process other than the zero-fill process.

Note that other preprocessing is performed as necessary before or after the zero-fill process is performed on the original image P2, but the description of other preprocessing is omitted here.

In this manner, training image PA2 can be created from the original image P2.

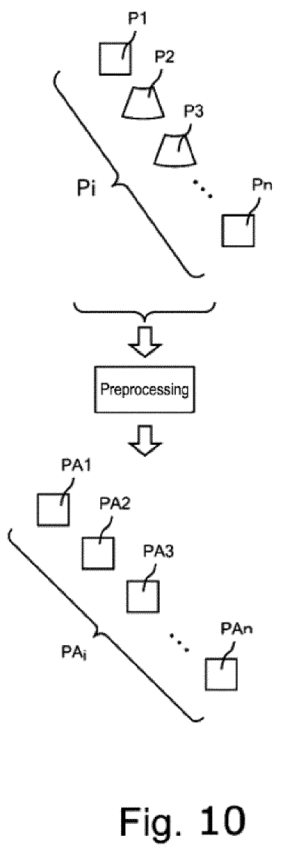

Similarly, preprocessing is performed on the other original images so that a square-shaped training image of 6 cm (length)×6 cm (width) is generated. Therefore, as shown in FIG. 10, training images PA1 to PAn created to have a common size can be prepared from the original images P1 to Pn.

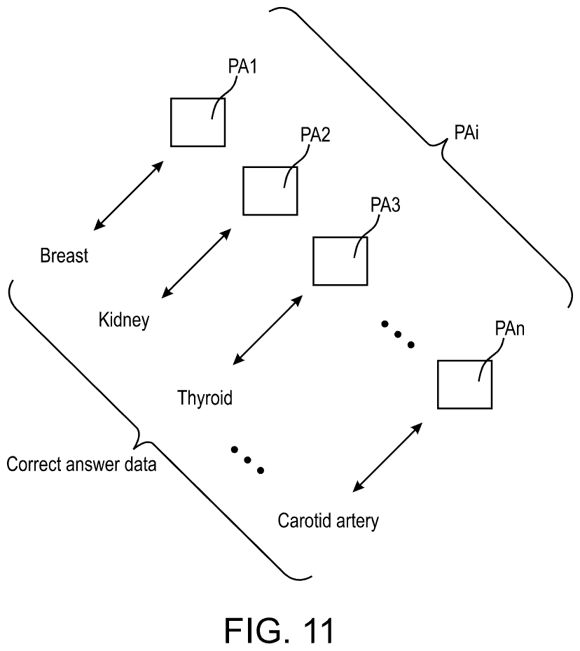

Next, the correct data is labeled on these training images PA1 to PAn (see FIG. 11).

FIG. 11 is an explanatory diagram of the correct data.

The training image PA1 is an image of a breast. Therefore, “breast” is labeled as the correct data in the training image PA1.

In addition, the training image PA2 is an image of a kidney. Therefore, “kidney” is labeled as the correct data in the training image PA2.

Similarly, the correct data is labeled for the other training images PA3 to PAn. Therefore, the correct data is labeled on all training images PA1 to PAn.

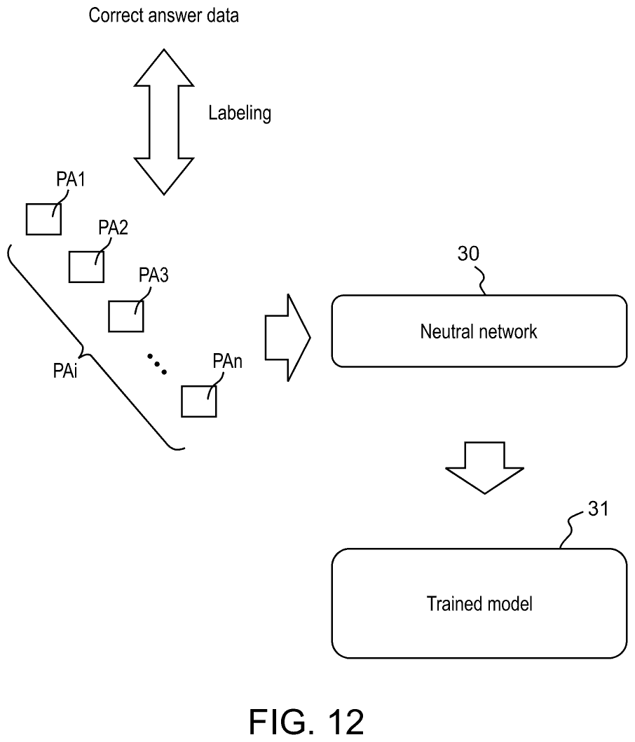

Next, as depicted in FIG. 12, the neural network 30 is trained with the above training images PA1 to PAn to create a trained model 31. The trained model 31 is stored in memory or external storage device. The trained model 31 can be created using any training algorithm used in AI learning, machine learning, or deep learning. For example, the trained model 31 may be created by supervised or unsupervised learning.

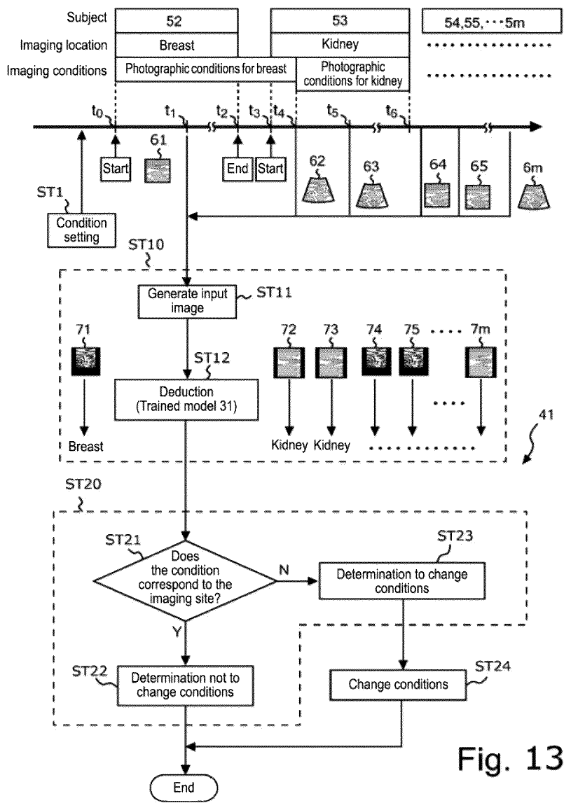

The first embodiment uses the trained model 31 to perform automatic changes to the imaging conditions. An example of how to automatically change the imaging conditions is described below, with reference to FIG. 13.

FIG. 13 is a diagram depicting an example of a flowchart executed during an examination of a subject.

In step ST1, the user 51 leads the subject 52 (see FIG. 1) to an examination room and has the subject 52 lie down on an examination bed.

The user 51 operates the user interface 10 (see FIG. 2) to enter patient information, set imaging conditions for acquiring ultrasonic images of the subject, and make other necessary settings. Note that the imaging conditions include any conditions related to the acquisition of ultrasonic images, such as the conditions for transmitting the ultrasonic beam, the conditions for receiving echoes from the subject, and the data processing conditions used to create an ultrasonic image based on the received echoes.

Here, the imaging site of the subject is set to the “breast”. Therefore, the user sets the imaging conditions for the breast.

When the user is ready for the examination, the user begins examining the subject 52. In FIG. 13, the inspection start time is indicated as t0.

Note that in FIG. 13, “subject”, “imaging site”, and “imaging conditions” are depicted on the time axis. The “subject” represents the subject being examined, “imaging site” represents the imaging site of the subject, and “imaging conditions” represents the imaging conditions set for the ultrasonic diagnostic device. For example, at time t0 when the examination starts, the diagram depicts that the “subject” is subject 52, the “imaging site” is the breast, and the “imaging conditions” are the imaging conditions for the breast.

The user 51 operates the probe and scans the subject 52 while pressing the ultrasonic probe 2 against an imaging site of the subject 52. Herein, the examination site is the mammary gland so, as illustrated in FIG. 1, the user 51 presses the ultrasonic probe 2 against the mammary gland of the subject 52. The ultrasonic probe 2 transmits an ultrasonic wave and receives an echo reflected from within the subject 52. The received echo is converted to an electrical signal, and this electrical signal is output as an echo signal to the receiving apparatus 5 (see FIG. 2). The receiver 5 executes a prescribed process on the echo signal and outputs the echo signal to the reception beamformer 6. The reception beamformer 6 executes reception beamforming on the signal received through the receiver 5 and outputs echo data.

The processor 7 generates an ultrasonic image based on the echo data. The ultrasonic image is displayed on the display unit 8.

The user 51 checks the ultrasonic image displayed on the display unit 8, and saves the ultrasonic image if necessary. Furthermore, the user 51 continues to perform the examination of the subject.

On the other hand, the processor 7 periodically executes a process 41 after the examination of subject starts at time t0 to determine whether the imaging conditions should be changed and to automatically change the imaging conditions as necessary. In this embodiment, the first process 41 is executed at time t1 after the inspection start time t0. The process 41 is described below.

When process 41 is initiated, first, in step ST10, the processor 7 identifies the imaging site in the ultrasonic image acquired between time points t0 and t1. The identifying step ST10 will be described below.

First, in step ST11, the processor generates an input image 71 for inputting to the trained model 31 based on an ultrasonic image 61 acquired between time t0 and time t1, and displayed on the display unit 8.

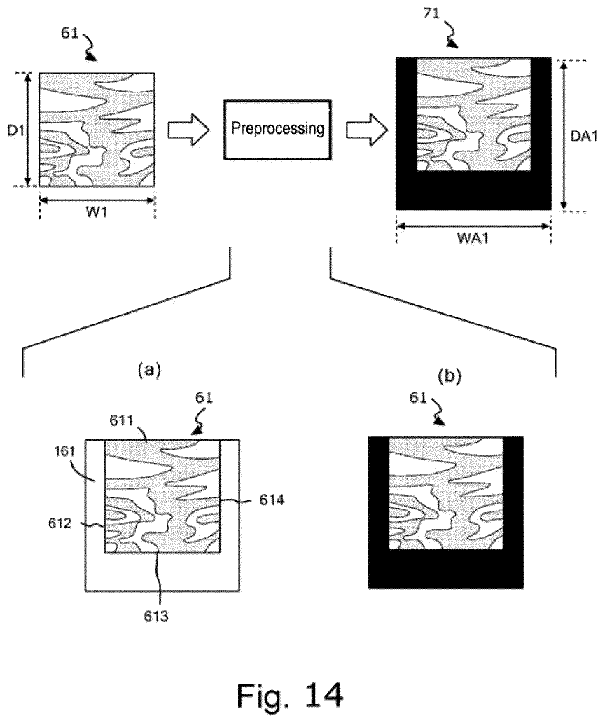

FIG. 14 is an explanatory diagram of step ST11.

If one ultrasonic image 61 is acquired between time t0 and time t1, the processor can generate an input image 71 in order to input to the trained model 31 based on the ultrasonic image 61. On the other hand, if a plurality of ultrasonic images have been acquired between time t0 and time t1, the processor selects one of the plurality of ultrasonic images 61 and can generate an input image 71 for inputting to the trained model 31 based on the selected ultrasonic image 61. If a plurality of ultrasonic images have been acquired between time t0 and time t1, the processor can typically select the last ultrasonic image acquired between time t0 and time t1 (the ultrasonic image acquired just before time t1) as the ultrasonic image 61.

Ultrasonic image 61 is a square-shaped image (D1=W1=4 cm). Therefore, the same preprocessing as the method of creating the training image PA1 described with reference to FIG. 7 is performed on the ultrasonic image 61 to generate the input image 71. Note that the size of the input image 71 is the same as the size of the training image PA1 described earlier (DA1=WA1=6 cm). Thus, the processor generates an input image 71 (DA1=WA1=6 cm) which is larger than the ultrasonic image 61 from the ultrasonic image 61 (D1=W1=4 cm). Specifically, preprocessing is performed as follows.

The processor performs a zero-fill process on the ultrasonic image 61, filling the blank region 161 around the ultrasonic image 61 with zero data to match the size of the input image 71. Here, the blank region 161 is set along three sides 612, 613, and 614 of the four sides 611 to 614 of the ultrasonic image 61. FIG. 14 (a) depicts a schematic view of the ultrasonic image 61 before zero-fill processing, and FIG. 14 (b) depicts a schematic view of the ultrasonic image 61 after zero-fill processing. Therefore, although the ultrasonic image 61 itself is smaller than the size of the input image 71, an input image 71 of the desired size can be created from the ultrasonic image 61 by performing the aforementioned zero-fill process as preprocessing of the ultrasonic image 61. In addition, an input image 71 of the desired size is created by performing the zero-fill process. However, if the input image 71 can have the desired size, preprocessing other than the zero-fill process may be performed.

Note that other preprocessing is performed as necessary before or after the zero-fill process is performed on the 61 ultrasonic images, but a description of other preprocessing is omitted here. After the input image 71 is generated, the process proceeds to step ST12.

In step ST12, the processor 7 deduces a location indicated by the input image 71 using the trained model 31.

The processor 7 inputs the input image 71 into the trained model 31 and uses the trained model 31 to deduce the sites contained in the input image 71. In the deduction step, the processor calculates the probability that each imaging site is included in the input image 71. Furthermore, the processor then deduces the imaging site in the input image 71 based on the probability calculated for each imaging site.

It can be assumed that the breast probability exceeds the threshold value. Therefore, the processor deduces that the imaging site included in the input image 71 is the breast. After deducing the imaging site, the process proceeds to step ST20.

In step ST20, the processor determines whether to change the conditions based on the deduced imaging site. Step ST20 will be described below in detail.

First, in step ST21, the processor determines whether the currently set imaging conditions are those corresponding to the imaging site deduced in step ST12. If the currently set imaging conditions are the imaging conditions corresponding to the imaging site deduced in step ST12, the processor proceeds to step ST22, but if the currently set imaging conditions are not the imaging conditions corresponding to the imaging site deduced in step ST12, the process proceeds to step ST23.

At time t1, the set imaging condition is the imaging condition for the breast. On the other hand, the imaging site deduced in step ST12 is the breast. Therefore, the currently set imaging conditions (imaging conditions for a breast) are those corresponding to the imaging site (breast) deduced in step ST12, so the process proceeds to Step ST22, and the processor 7 determines not to change the imaging conditions, and terminates the process 41.

On the other hand, the user 51 continues the examination of the subject 52 while operating the ultrasonic probe 2 after time t1. Furthermore, after time t1, the processor continues to periodically execute the aforementioned process 41. Here, it can be assumed that the process 41 was performed after time t1, but it was determined (step ST22) not to change the imaging conditions. Therefore, the examination of the breast of the subject was completed without any automatic changes in the imaging conditions being made. The end point of the mammary gland imaging of the subject is indicated by “t2”. The user prepares the next new subject for examination.

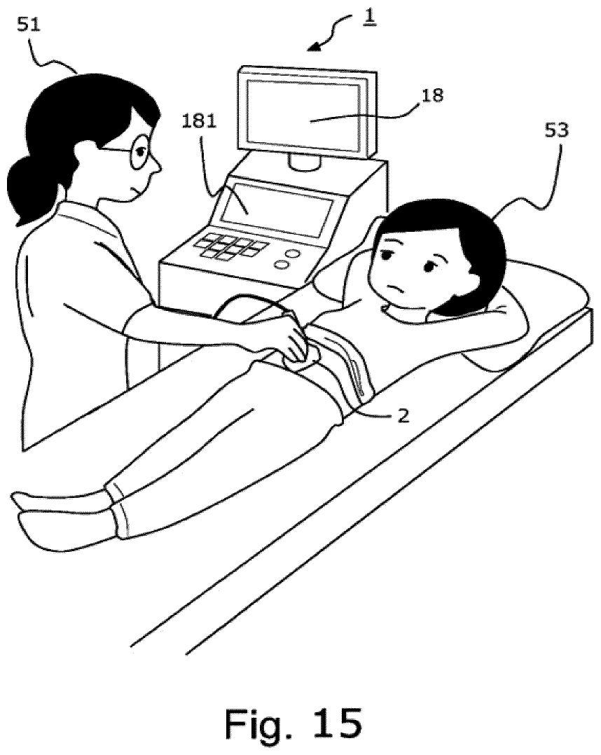

FIG. 15 depicts the next new subject to be examined.

The case where the imaging site of a new subject 53 is different from that of the immediately preceding subject 52 is described below. Here, the case where the imaging site of the immediately preceding subject 52 is the breast was described, but the imaging site of the new subject 53 is the kidney.

The user prepares for the examination of the kidney of the new subject 53 after completing the breast examination of the immediately preceding subject 52. In this case, the imaging site is changed from the breast to the kidney, so the user must change the imaging conditions from imaging conditions for the breast to the imaging conditions for the kidney. In the following, however, the case is considered in which the user initiates an examination of the kidney of a new subject 53 without changing the imaging conditions.

The user begins examining the kidneys of the subject 53 at time t3.

The user 51 has started examining the kidneys of the subject 53 from time t3, but has not changed the imaging conditions, so the set imaging conditions remain the same as those for the breast. Therefore, the user begins the examination of the kidney of the subject 53 under the imaging conditions for the breast. The user 51 presses the probe 52 against the abdomen of the subject 53 to examine the kidney, as depicted in FIG. 15.

On the other hand, the processor 7 periodically executes the process 41 after the examination of the kidney of the subject 53 begins at time t3. The present embodiment describes the case where the process 41 is executed at time t4 after time t3.

When process 41 is initiated, first, in step ST10, the processor 7 identifies the imaging site in the ultrasonic image acquired between time points t3 and t4. The identifying step ST10 will be described below.

First, in step ST11, the processor preprocesses the ultrasonic images 62 acquired between time t3 and time t4 and displayed on the display 2 to generate the input images 72 for input to the trained model 31.

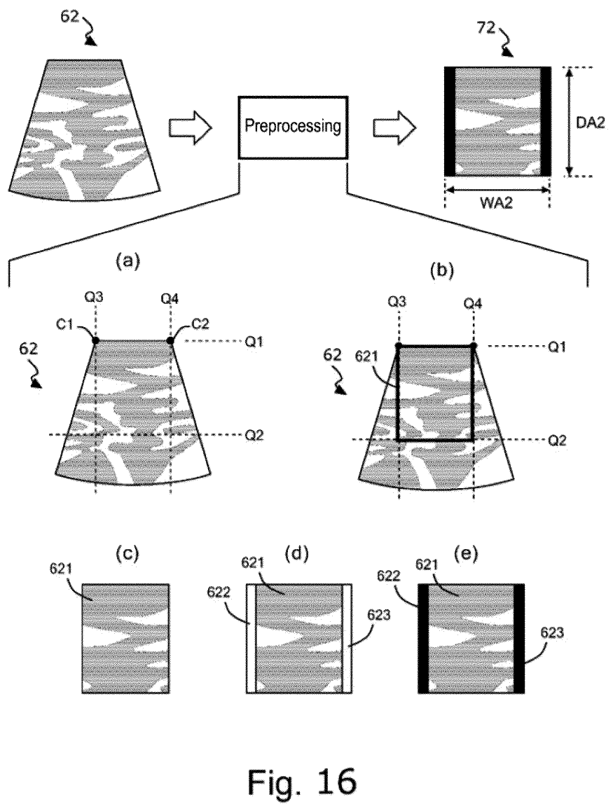

FIG. 16 is an explanatory diagram of step ST11.

If one ultrasonic image 62 is acquired between time t3 and time t4, the processor can generate an input image 72 in order to input to the trained model 31 based on the ultrasonic image 62. On the other hand, if a plurality of ultrasonic images have been acquired between time t3 and time t4, the processor selects one of the plurality of ultrasonic images 62 from the plurality of ultrasonic images, and can generate an input image 72 for inputting to the trained model 31 based on the selected ultrasonic image 62. If a plurality of ultrasonic images have been acquired between time t3 and time t4, the processor can typically select the last ultrasonic image acquired between time t3 and time t4 (for example, the ultrasonic image acquired just before time t4) as the ultrasonic image 62.

The ultrasonic image 62 is an essentially trapezoidal image. Accordingly, the processor performs the same preprocessing on the ultrasonic image 62 as the method of creating a training image described with reference to FIG. 9, and generates the input image 72. Note that the size of the input image 72 is the same as the size of the training image PA2 described earlier (DA2=WA2=6 cm). Thus, the processor generates a rectangular-shaped input image 72 from an essentially trapezoidal-shaped ultrasonic image 62. Specifically, preprocessing is performed as follows.

As depicted in FIG. 16 (a), with respect to the depth direction of the subject, the processor uses the region from position Q1 on the body surface of the ultrasonic image 62 to position Q2, which is only 6 cm away in the depth direction, as the image portion used to create the input image. Furthermore, with respect to the width direction, the region from position Q3 in the upper left corner C1 to position Q4 in the upper right corner C2 of the ultrasonic image 62 is the image portion used for the training image.

Therefore, the processor determines the region 621 defined by Q1 to Q4 in the ultrasonic image 62 as the image portion to be used to create the input image, as depicted in FIG. 16 b), and extracts the image portion 621 from the ultrasonic image 62 (see FIG. 16(c)).

Next, the processor performs a zero-fill process on the extracted image 621 cut from the ultrasonic image 62, filling the blank regions 622 and 623 along the side edges of the extracted image 621 with zero data to match the size of the input image 72. FIG. 16 (d) depicts a schematic diagram of the extracted image 621 before zero-filling the blank regions 622 and 623, and FIG. 16(e) depicts a schematic diagram of the extracted image 621 after zero-filling the blank regions 622 and 623. Thus, an input image 72 of the desired size can be created from the ultrasonic image 62. In addition, an input image 72 of the desired size is created by performing the zero-fill process. However, if the input image 72 can have the desired size, preprocessing other than the zero-fill process may be performed.

Note that other preprocessing is performed as necessary before or after the zero-fill process is performed on the 62 ultrasonic images, but a description of other preprocessing is omitted here. After the input image 72 is generated, the process proceeds to step ST12.

In step ST12, the processor 7 deduces a location indicated by the input image 72 using the trained model 31.

The processor 7 inputs the input image 72 into the trained model 31 and uses the trained model 31 to deduce the sites contained in the input image 72. In the deduction step, the processor calculates the probability that each imaging site is included in the input image 72. Furthermore, the processor then deduces the imaging site in the input image 72 based on the probability calculated for each imaging site.

Here, it is assumed that the probability of kidney is highest. Therefore, the processor deduces that the imaging site included in the input image is the kidney. After deducing the imaging site, the process proceeds to step ST20.

In step ST20, the processor determines whether to change the conditions based on the deduced imaging site. Step ST20 will be described below in detail.

First, in step ST21, the processor determines whether the currently set imaging conditions are those corresponding to the imaging site deduced in step ST12. If the currently set imaging conditions are the imaging conditions corresponding to the imaging site deduced in step ST12, the processor proceeds to step ST22, but if the currently set imaging conditions are not the imaging conditions corresponding to the imaging site deduced in step ST12, the process proceeds to step ST23.

At time t4, the set imaging condition is the imaging condition for the breast. On the other hand, the imaging site deduced in step ST12 is the kidney. Therefore, currently set imaging conditions (imaging conditions for the breast) are not the imaging conditions corresponding to the imaging site (kidney) deduced in step ST12, so the process proceeds to step ST23.

In step ST23, the processor makes a determination to change the imaging conditions. Next, the process proceeds to step ST24 to change the imaging conditions from the imaging conditions for the breast to the imaging conditions for the kidney. FIG. 13 depicts the change to imaging conditions for the kidney immediately after time t4.

Thus, the user begins imaging the kidney under breast imaging conditions, but the processor automatically changes the imaging conditions to kidney imaging conditions during the course of imaging of the kidney by the user. Therefore, even if the user forgets to change the imaging conditions, after the processor changes the imaging conditions, the user can still acquire high-quality kidney images because the user can still image the kidney according to the kidney imaging conditions.

On the other hand, the user 51 continues to examine the kidney of the subject 53 while operating the ultrasonic probe 2 after time t4, and the processor periodically executes the above process 41. Here, at time t5 after time t4, the flow of process 41 is performed.

When the flow of process 41 starts at time t5 in step st11, an input image 73 is generated by preprocessing the ultrasonic image 63 displayed on the display unit 8 using the method depicted in FIG. 9. In step ST12, the input image 73 is input to the trained model 31 to deduce the imaging site. Next, in step ST20, a determination is made as to whether or not to change the imaging conditions, and the flow is terminated.

On the other hand, the user 51 continues the examination of the subject 53 while operating the ultrasonic probe 2 after time t5. After time t5, the processor periodically executes the aforementioned process 41. In this case, a decision is made not to change the imaging conditions in process 41 after time t5 (step ST22), and the examination of the subject 53 is completed at time t6.

Once the examination of subject 53 is completed, the flow of process 41 is periodically performed on the next new subject to be examined. Similarly, the flow of process 41 is periodically performed when each new subject is tested.

FIG. 13 schematically depicts how the process 41 is performed periodically, even after examination of the subject 53. Specifically, after time t6 in step ST11, input images 74, 75, . . . 7m are generated by preprocessing the ultrasonic images 64, 65, . . . 6m displayed on the display unit 8, and in step ST12, the imaging sites in the input images 74, 75, . . . 7m are deduced. Next, in step ST20, a determination is made as to whether to change the imaging conditions, the imaging conditions are changed if necessary (step ST24), and the flow of process 41 ends.

As described above, in the present embodiment, input images 71 to 7m are generated to have a predetermined size regardless of the imaging conditions of the ultrasonic images 61 to 6m. Therefore, even if the user (or processor) changes the ultrasonic viewing angle or the ultrasonic depth depending on the imaging site of the subject, or even if the user (or processor) changes the viewing angle of the ultrasonic or the depth of the ultrasonic while the subject is being examined, in step ST11, input images 71 to 7m with a predetermined size will be obtained. Thus, for example, even if the processor changes the imaging conditions at time t4 or the user manually changes the ultrasonic depth or other parameters during the examination of the subject 52 (or 53), input images 71 to 7m with a predetermined size are obtained in step ST11. Therefore, in step ST12, the processor deduces based on same size input images 71 to 7m, which improves the accuracy of identification of the imaging site and provides stable deduction results.

In the present embodiment, the length in the depth direction of the input image is the length measured from the body surface of the subject. However, if an input image of a predetermined size is to be generated, the depth length of the input image may be set as the length measured from a reference plane other than the body surface of the subject (for example, a plane contained within the subject or the surface of an organ).

Note that in the present embodiment, as depicted in FIG. 14, the blank region 161 is set along three sides 612, 613, and 614 of the four sides 611 to 614 of the ultrasonic image 61. However, the blank region does not necessarily need to be set along the three sides, and various blank regions can be set.

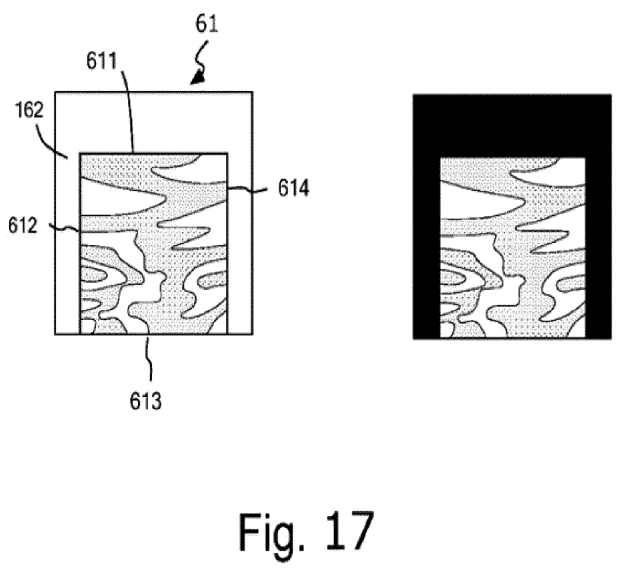

FIG. 17 to FIG. 19 are diagrams depicting a variation of the blank region.

FIG. 17 depicts an example where the blank region 162 is set along the three sides 611, 612, and 614 of the ultrasonic image 61. Therefore, a zero-fill process is performed in which the blank regions 162 along the three edges 611, 612, and 614 are filled with zeros.

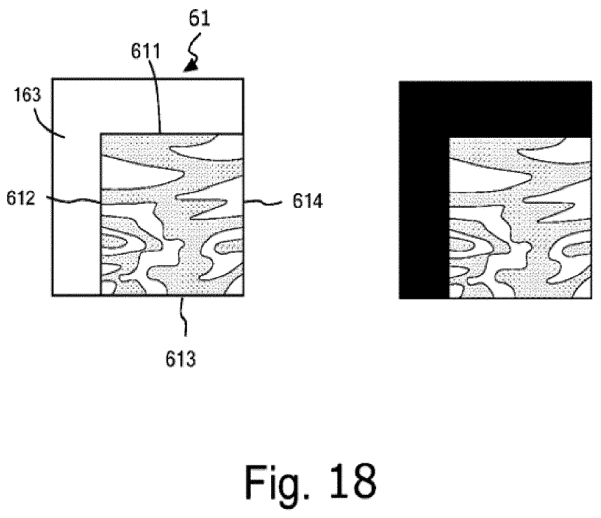

FIG. 18 depicts an example where the blank region 163 is set along two sides 613 and 614 of the ultrasonic image 61. Thus, a zero-fill process is performed in which the blank regions 163 along the two sides 613 and 614 are filled with zeros.

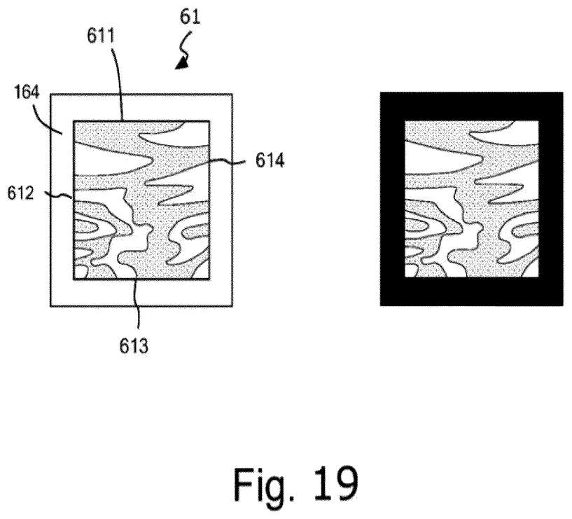

FIG. 19 depicts an example where the blank regions 164 are set along the four sides 611 to 614 of the ultrasonic image 61. Thus, a zero-fill process is performed in which the blank regions 164 along the four sides 611 to 614 are filled with zeros.

Thus, if it is possible to create an input image with the desired size, the blank region can be set to any shape of ultrasonic image.

Furthermore, in the present embodiment, as depicted in FIG. 16, the extracted image 621 is extracted based on position Q1 on the body surface of the ultrasonic image 62. However, the extracted image can be extracted based on any position in the ultrasonic image 62.

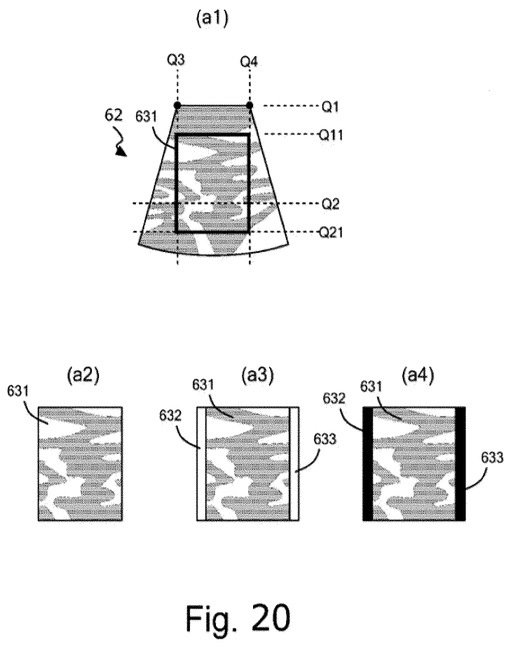

FIG. 20 is a diagram depicting a variation of the extracted image.

In FIG. 20, as depicted in schematic diagram (al), an example is depicted where the image portion used for the training image is the region from position Q11, which is lower than position Q1 in the depth direction, to position Q21, which is 6 cm lower in the depth direction. Thus, the region bounded by positions Q11, Q21, Q3, and Q4 is the extracted image 631 (see schematic diagram (a2)). Furthermore, blank regions 632 and 633 are set on the side edges of the extracted image 631 (schematic diagram (a3)) and zero-fill processing is performed (schematic diagram (a4)). Thus, the reference position for extracting the extracted image does not necessarily have to be the body surface, and the image can be extracted based on a desired position corresponding to the imaging conditions and the like of the ultrasonic image.

What is claimed is:

1. An ultrasonic diagnostic device, comprising:
an ultrasonic probe;
a display unit; and
a processor configured to communicate with the ultrasonic probe and the display unit;
wherein the processor is configured to:
set conditions for acquiring ultrasonic images of the subject;
transmit an ultrasonic beam to the ultrasonic probe and causing the ultrasonic probe to receive an echo from the subject in accordance with the conditions;
generate an ultrasonic image of the subject based on the echo received by the ultrasonic probe; and
create input images to be input to the trained model based on the ultrasound images displayed on the display unit;
wherein each input image created by the processor is created such that the length of the subject in the depth direction is a first length, and the length of the subject in a direction perpendicular to the depth direction is a second length.

2. The ultrasonic diagnostic device according to claim 1, wherein
creating an input image based on the ultrasonic image includes performing preprocessing on the ultrasonic image.

3. The ultrasonic diagnostic device according to claim 2, wherein the processor is further configured to, based on the length of the ultrasonic image in the depth direction of the subject being greater than a predetermined length:
extract a portion of the ultrasound image during the preprocessing, and
create the input image based on the extracted image.

4. The ultrasonic diagnostic device according to claim 3, wherein
the preprocessing includes performing predetermined processing on the extracted image such that an input image of a desired size is created.

5. The ultrasonic diagnostic device according to claim 4, wherein the predetermined processing includes zero-fill processing.

6. The ultrasonic diagnostic device according to claim 2, wherein the processor is further configured to, based on the length of the ultrasonic image in the depth direction of the subject being less than the predetermined length, perform predetermined processing on the ultrasonic image so that an input image of the desired size is created.

7. The ultrasonic diagnostic device according to claim 6, wherein
the predetermined processing includes zero-fill processing.

8. The ultrasonic diagnostic device according to claim 1, wherein
the first length is the length between the position on the body surface of the subject and a position inside the body of the subject.

9. The ultrasonic diagnostic device according to claim 1, wherein the second length is equal to the first length.

10. The ultrasonic diagnostic device according to claim 1, wherein
a trained model is created by a neural network learning from a plurality of training images,
and each training image has a first length in the depth direction of the subject and a second length in a direction orthogonal to the depth direction of the subject.

11. The ultrasonic diagnostic device according to claim 1, wherein
the processor is configured to the input image to the trained model and deduce a site included in the input image using the trained model.

12. A non-transitory computer readable storage medium for storing commands that when executed by a processor cause the processor to:
set conditions for acquiring ultrasonic images of the subject;
transmit an ultrasonic beam to the ultrasonic probe and causing the ultrasonic probe to receive an echo from the subject in accordance with the conditions;
generate an ultrasonic image of the subject based on the echo received by the ultrasonic probe; and
create input images to be input to the trained model based on the ultrasonic images displayed on the display unit;
wherein each input image created by the processor is created such that the length of the subject in the depth direction is a first length, and the length of the subject in a direction perpendicular to the depth direction is a second length.

13. A method, comprising:
setting conditions of an ultrasonic probe for acquiring ultrasonic images of the subject;
transmitting an ultrasonic beam from the ultrasonic probe towards the subject;
receiving, by the ultrasonic probe, an echo of the ultrasonic beam from the subject in accordance with the conditions;
generating an ultrasonic image of the subject based on the echo received by the ultrasonic probe; and
creating input images to be input to the trained model based on the ultrasonic images displayed on the display unit;
wherein each input image created by the processor is created such that the length of the subject in the depth direction is a first length, and the length of the subject in a direction perpendicular to the depth direction is a second length.
